(19) United States
(12) Patent Application Publication
(10) Pub. No.: US 2008/0149111 A1
(43) Pub. Date: Jun. 26, 2008

(54) COLLAPSIBLE, DISPOSABLE, ADJUSTABLE STERILITY MAINTENANCE COVER

(76) Inventors: Samuel W. Harrison, Shreveport, LA (US); Patrick Quick, Stonewall, LA (US)

Correspondence Address: MARK YOUNG, P.A., 12086 FORT CAROLINE ROAD, UNIT 202, JACKSONVILLE, FL, 32225, US

(21) Appl. No.: 11/615,401

(22) Filed: Dec. 22, 2006

(51) Int. Cl. A61B 19/10; B65D 69/00; B65D 85/00

(52) U.S. Cl. 128/852; 206/570; 206/370

(57) ABSTRACT

A sterility maintenance cover is equipped with an instrument tray. The tray may be pivotally mounted hingedly attached, removable, stationary an/or adapted for height adjustment and leveling. Clamps may be provided for releasably engaging the removable tray. The sterility maintenance cover may be formed from a unitary blank of corrugated material.

### FIELD OF THE INVENTION

This invention generally relates to surgical procedure devices, and more particularly, to a collapsible, disposable, adjustable sterility maintenance cover with a platform designed to support a drape over a patient's head and support various instruments used in a surgical procedure.

### BACKGROUND

During certain surgical procedures, such as pacemaker implants and similar procedures, it is imperative that a patient's head be draped in a sterile manner to insure that a sterile field is maintained throughout the operation. Various methods have been employed to achieve this result, including various types of draping techniques, using supports and other devices. For example, U.S. Pat. No. 5,322,072 to Harrison et al. and U.S. Pat. No. 5,546,961 to Harrison teach sterility maintenance covers that support a surgical drape over a patient's head.

A sterility maintenance cover generally includes (i) a base panel for fitting under the head, neck and shoulders of a patient positioned on an examination or operating table, (ii) an upward-standing end panel extending from the base panel and having an open window for accessing the patient from the end or side of the table, and (iii) a cantilevered platform projecting horizontally from the end panel, substantially parallel to the base panel, over the patient's head and neck. The platform supports a drape over a patient's head and/or serves as a tray that receives and retain or deploy various instruments used in the procedure. A sterility maintenance cover may also include one or more windows to facilitate access to and observation of the patient's head.

Unfortunately, conventional sterility maintenance covers have several shortcomings. One shortcoming is that they are reusable non-disposable designs. Thus, conventional sterility maintenance covers must be sterilized between surgical procedures to avoid contamination. Another shortcoming is that conventional sterility maintenance covers are non-collapsible, 3-D unitary structures, which are cumbersome to manufacture, ship and store. Yet another shortcoming is that conventional sterility maintenance covers are rigid non-adjustable structures. Thus, the tray cannot be raised, lowered or pivoted to facilitate access to a patient and performance of a procedure.

Accordingly, a need exists for a collapsible, disposable, adjustable sterility maintenance cover with a platform designed to support a drape over a patient's head and support various instruments used in a surgical procedure. The invention is directed to overcoming one or more of the problems and solving one or more of the needs as set forth above.

### SUMMARY OF THE INVENTION

In one embodiment of the invention, a sterility maintenance cover according to principles of the invention includes a base panel, a first vertical support, an instrument tray removably attached to the first vertical support at the top end. The first vertical support has a bottom end and a top end. The first vertical support is attached to the base panel at the bottom end and extends upwardly from the base panel to a height sufficient to clear a patient's anatomical structure. The instrument tray has a topside with a periphery has raised edges.

Optionally, in a preferred implementation, the sterility maintenance cover further includes a support flange extending outwardly from the top end of the first vertical support. The support flange is configured to abut and provide support to the instrument tray.

The sterility maintenance cover may further include attachment means adapted to removably and pivotally attach the instrument tray to the first vertical support. The attachment means may include a screw adapted to extend through an aperture with a grommet in the instrument tray and thread into a threaded hole in the top end of the first vertical support.

In another embodiment, the sterility maintenance cover includes first and second vertical supports. The second vertical support has a bottom end and a top end. The second vertical support is attached to the base panel at the bottom end extending upwardly from the from the base panel to a height sufficient to clear a patient's anatomical structure. The second vertical support structure is a determined distance (e.g., approximately the length of an edge) from the first vertical support structure. The sterility maintenance cover further includes attachment means adapted to removably and pivotally attach the instrument tray to the first vertical support and second vertical support.

Optionally, in a preferred implementation, the sterility maintenance cover further includes a plurality of support flanges. At least one of the plurality of support flanges extending outwardly from the top end of each of the first and second vertical supports and is configured to abut and provide support to the instrument tray.

In yet another embodiment, the sterility maintenance cover includes a clamp attached to the top end of each vertical support. The clamp is adapted to securely releasably engage an edge of the instrument tray. The clamp may include a protrusion configured to resist removal of the instrument tray after engagement.

In still another embodiment, the sterility maintenance cover includes a height adjustment means adapted to enable manual adjustment of the length of each of the first and second vertical supports. The height adjustment means may comprise a manually adjustable first threaded shaft operably coupled to the first vertical support, and a manually adjustable second threaded shaft operably coupled to the second vertical support. A level indicator (e.g., a spirit level) may be operably coupled to the instrument tray.

The base panel, first vertical support and instrument tray may be comprised of a transparent radiolucent plastic. The plastic may include an additive, such as a thermochromic, photochromic or phosphorescent additive.

In a further embodiment, the sterility maintenance cover includes a base panel, an instrument tray, a first vertical support with a bottom end and a top end, a second vertical support with a bottom end and a top end, and a plurality of hinged attachments. The base panel is hingedly attached to the bottom end of the first and second vertical supports. The instrument tray is hingedly attached to the top end of the first and second vertical supports, and has a topside with a periphery having raised edges. The base panel and instrument tray are adapted to hingedly pivot between an operational configuration and a storage configuration. The operational configuration is weight-bearing and perpendicular to the longitudinal axes of the first and second vertical supports.

In yet another further embodiment, the sterility maintenance cover is comprised of a base panel formed from an integral blank (i.e., a unitary sheet) having a first, second, third and fourth panels side-by-side. A first vertical support is formed from the second panel and has a bottom end and a top end. An instrument tray is formed from the third panel. A second vertical support is formed from the fourth panel. A second vertical support has a bottom end and a top end and is formed from the fourth panel. A base panel is formed from the first panel. The integral blank may be comprised of corrugated plastic, corrugated paperboard, fluted corrugated plastic, fluted corrugated paperboard, or micro-corrugated paperboard.

### BRIEF DESCRIPTION OF THE DRAWINGS

The foregoing and other aspects, objects, features and advantages of the invention will become better understood with reference to the following description, appended claims, and accompanying drawings, where:

FIG. 1 is a perspective view of an exemplary sterility maintenance cover with a pivotally adjustable tray in accordance with principles of the invention; and

FIG. 2 is a perspective view of an exemplary modular sterility maintenance cover in accordance with principles of the invention; and

FIG. 3 is a perspective view of an exemplary modular sterility maintenance cover with support flanges in accordance with principles of the invention; and

FIG. 4 is a perspective view of an exemplary sterility maintenance cover with clamps for engaging a removable tray in accordance with principles of the invention; and

FIG. 5 is a plan view of a blank for creating an exemplary sterility maintenance cover in accordance with principles of the invention; and

FIG. 6 is a perspective view of an exemplary sterility maintenance cover created from a blank as shown in FIG. 5 in accordance with principles of the invention; and

FIG. 7 is a perspective view of an exemplary sterility maintenance cover with an adjustable height tray for engaging a removable tray in accordance with principles of the invention; and

FIG. 8A is a perspective view of an exemplary sterility maintenance cover with a hinged tray and hinged base configured in an operational orientation in accordance with principles of the invention; and

FIG. 8B is a perspective view of an exemplary sterility maintenance cover with a hinged tray and hinged base configured in a storage orientation in accordance with principles of the invention; and

FIG. 9 is a perspective view of a patient with an exemplary sterility maintenance cover configured in an operational position in accordance with principles of the invention.

Those skilled in the art will appreciate that the invention is not limited to the exemplary embodiments depicted in the figures or the shapes, relative sizes, proportions or materials shown in the figures.

### DETAILED DESCRIPTION

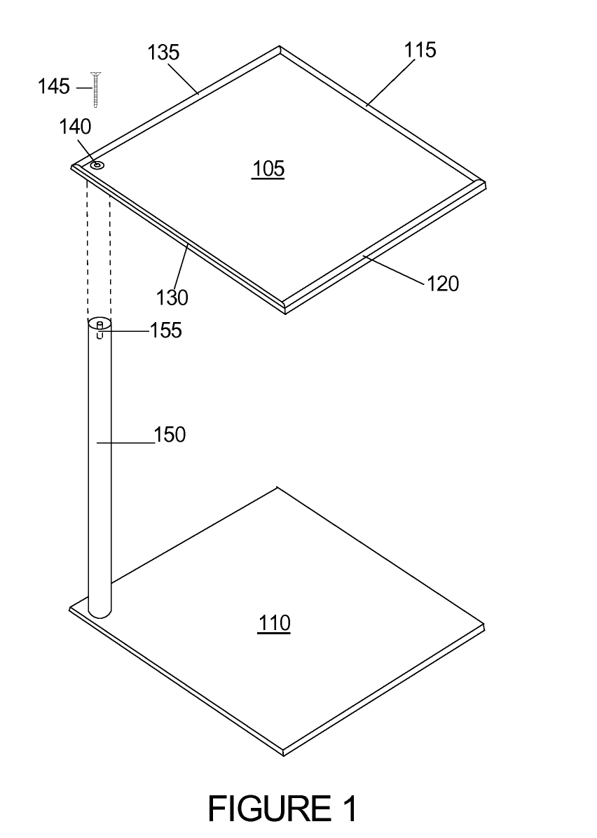

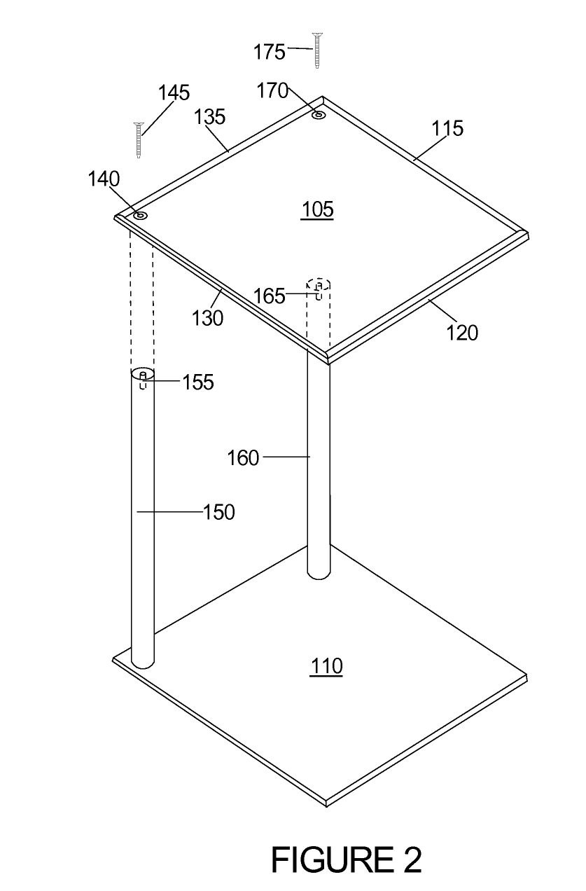

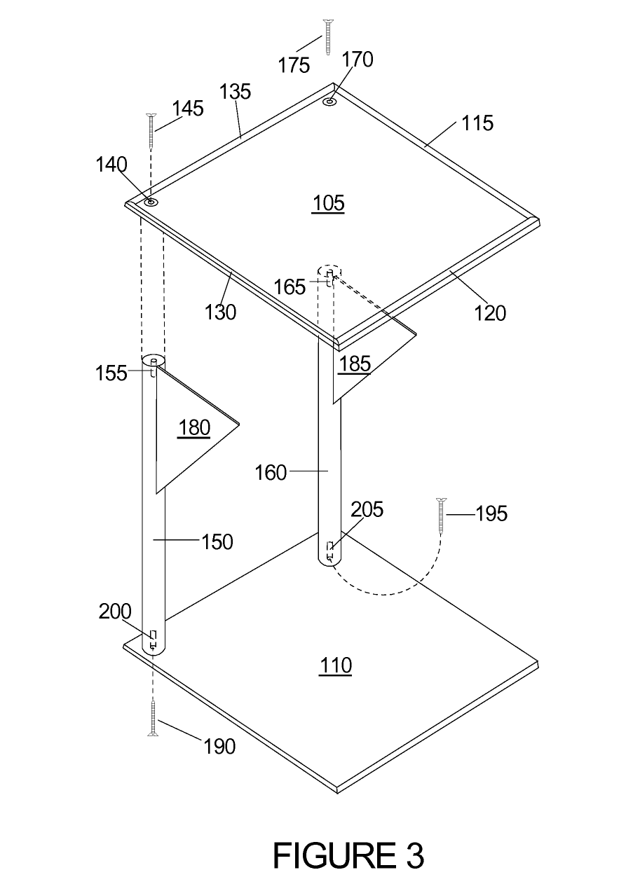

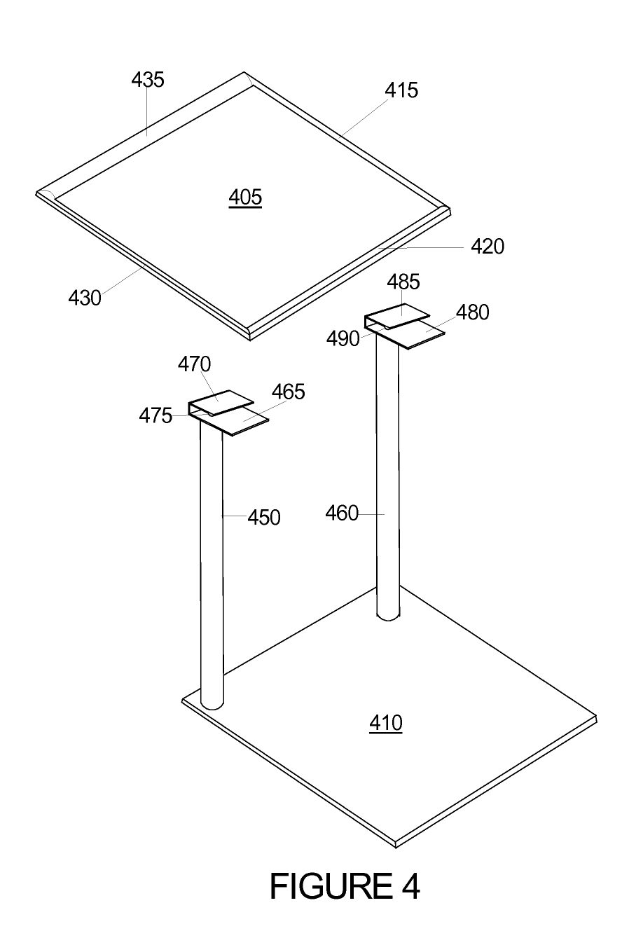

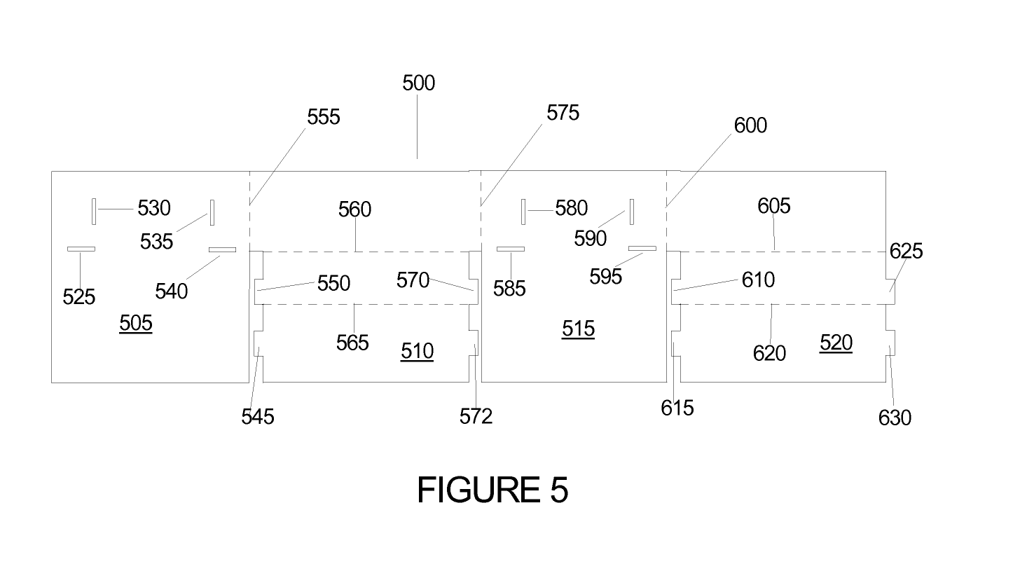

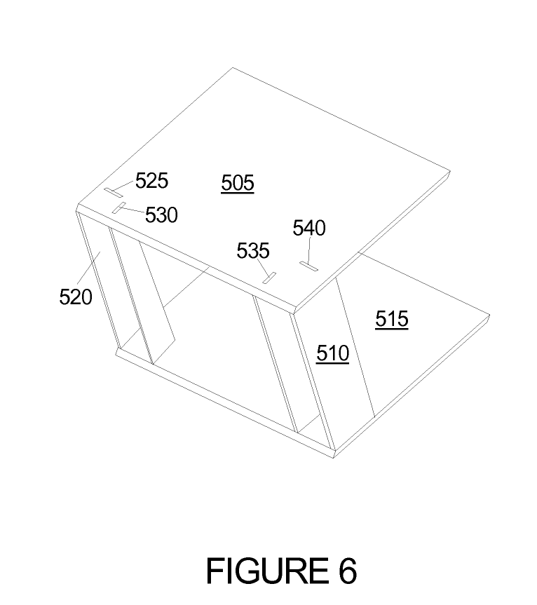

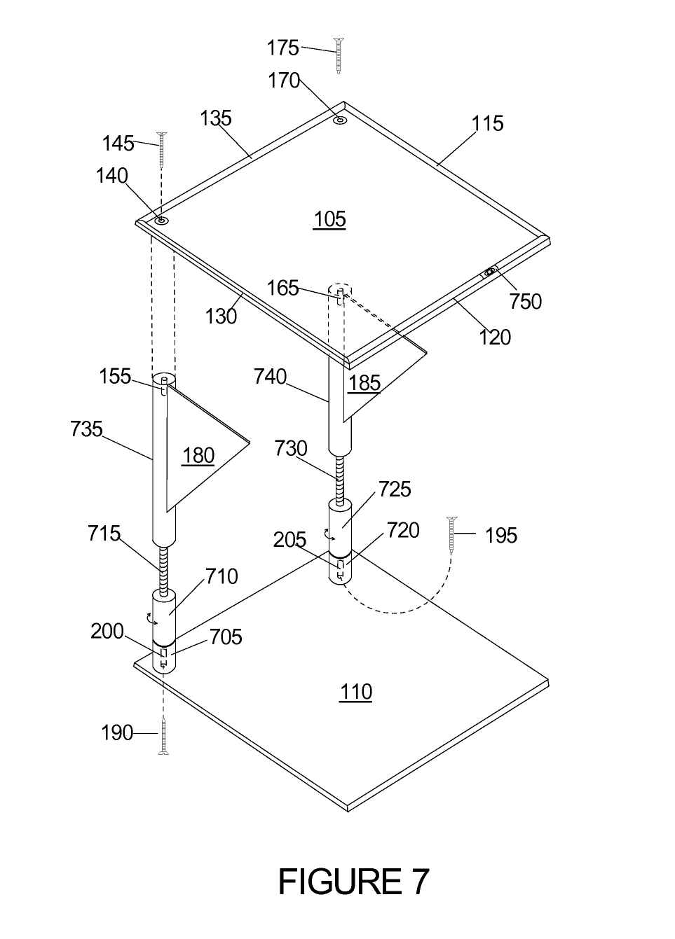

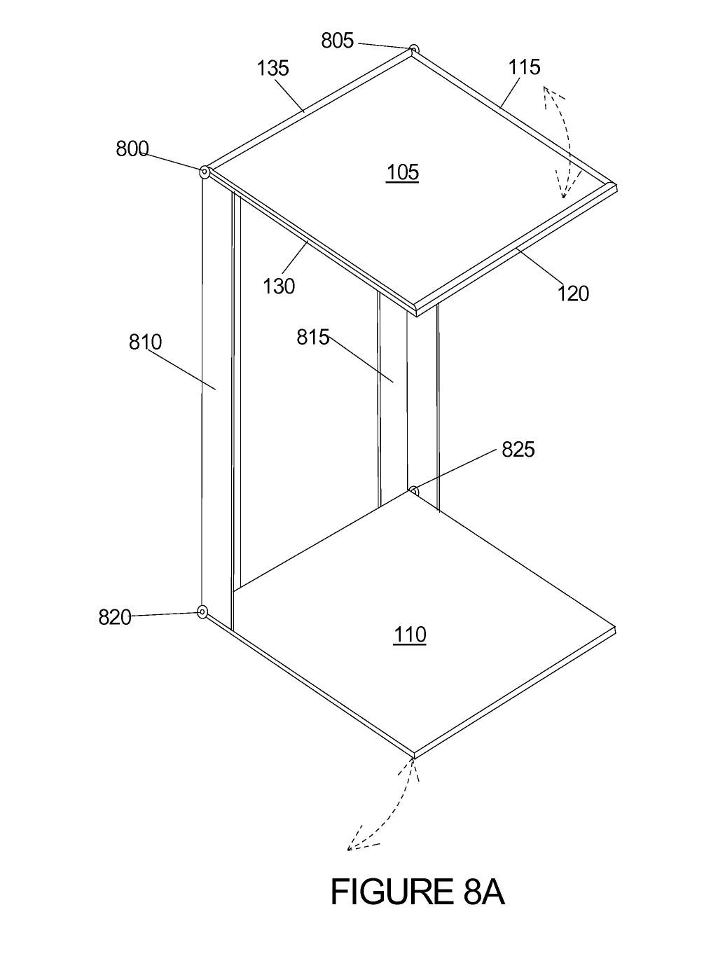

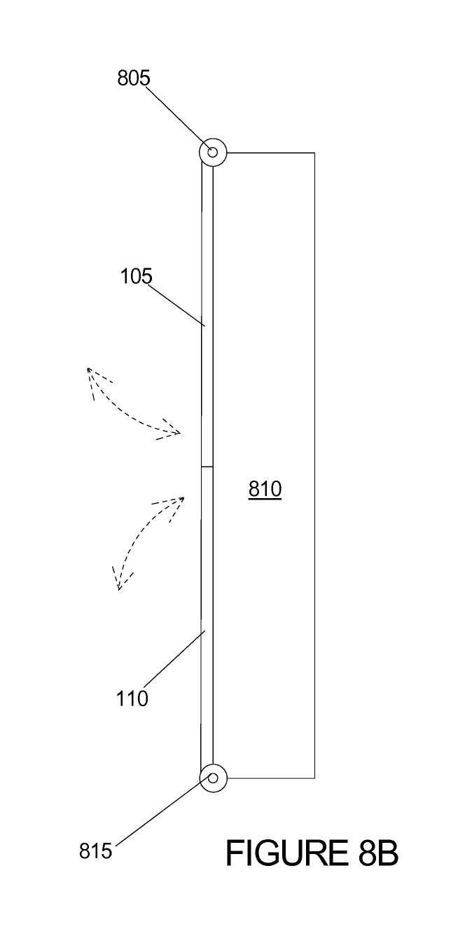

With reference to the drawings, wherein like numerals represent like features, exemplary embodiments of sterility maintenance covers in accordance with principles of the invention are conceptually shown. The exemplary embodiments include a sterility maintenance cover with a pivotally adjustable removable tray as in FIG. 1, a sterility maintenance cover with a stationary removable tray as in FIG. 2 and FIG. 3, a sterility maintenance cover with clamps for engaging a removable tray as in FIG. 4, a unitary blank for forming a sterility maintenance cover as in FIG. 5, a sterility maintenance cover formed from the unitary blank as in FIG. 6, a sterility maintenance cover with a height adjustable tray as in FIG. 7, and a sterility maintenance cover with a hinged tray and hinged base configured for folding from storage to operational positions as in FIG. 8. Each exemplary embodiment is described more fully below.

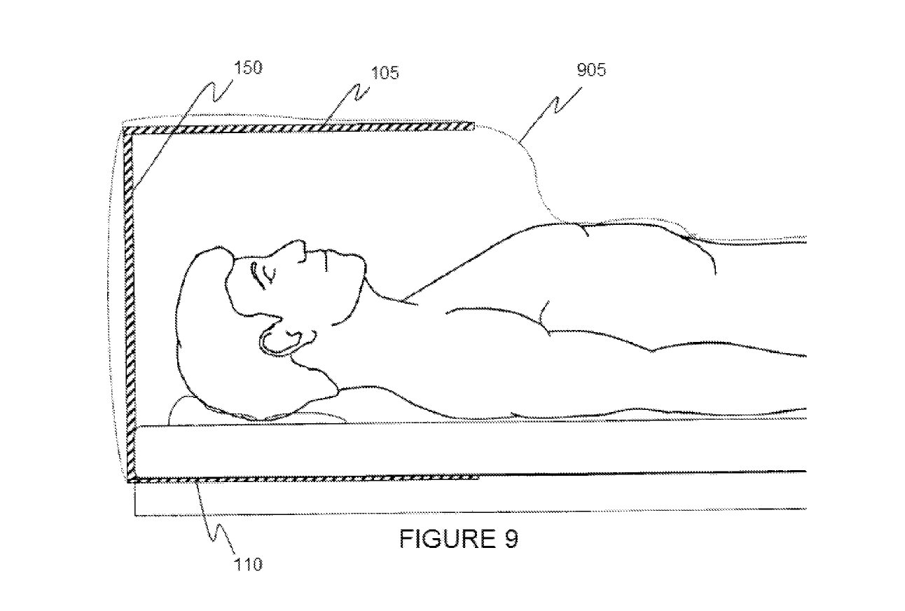

Referring now to FIG. 1, a sterility maintenance cover with a pivotally adjustable removable tray is shown. The sterility maintenance cover is designed not only for maintaining sterility during certain types of operating procedures, but also to support surgical and other instruments in an efficient manner, in emergency room, intensive care, operating room, home health care and examination room applications. The sterility maintenance cover generally includes a base panel 110 configured to provide a stable support surface. The base panel is adapted for fitting on a flat surface such as an examination or operating table and selectively under the head, neck and shoulders, as well as the torso, thighs and feet of a patient, as conceptually shown in FIG. 9. A vertical support 150 extends from the base panel 110 to a height sufficient to clear a patient's head or other anatomical structure. An instrument tray 105 is removably attached to the vertical support 150 at the end opposite the base panel 110. The periphery of the topside of the instrument tray 105 includes raised edges 115-135 to prevent objects from sliding off.

Attachment means is provided to removably and pivotally attach the instrument tray 105 to the vertical support 150. By way of example and not limitation, the attachment means may comprise a screw 145 adapted to extend through an aperture with a grommet 140 in the instrument tray 105 and thread into a threaded hole 155 in the vertical support. Loosening the screw 145 facilitates pivotally adjusting the angular position of the instrument tray 105 relative to the base panel 110. Tightening the screw 145 secures the instrument tray 105 in a position. Removal of the screw 145 allows disassembly of the sterility maintenance cover and removal of the instrument tray 105. Although not shown in FIG. 1, the bottom end of the vertical support 150 may be removably attached to the base panel in a similar manner. Other forms of permanent or releasable attachment may be utilized in addition to or in lieu of screws.

Referring now to FIG. 2, an exemplary modular sterility maintenance cover in accordance with principles of the invention is shown. This embodiment is similar to the embodiment depicted in FIG. 1, except it includes a plurality of vertical supports 150, 160 for enhanced structural integrity. The sterility maintenance cover generally includes a base panel 110 configured to provide a stable support surface. A plurality of vertical supports 150, 160 extend from the base panel 110 to a height sufficient to clear a patient's head or other anatomical structure. Advantageously, the upward-standing vertical supports 150, 160 extending from the base panel provide minimal obstruction to the patient. The instrument tray 105 is removably attached to the vertical supports 150, 160 at the end opposite the base panel 110. The periphery of the topside of the instrument tray 105 includes raised edges 115-135 to prevent objects from sliding off.

Attachment means is provided to removably attach the instrument tray 105 to the vertical supports 150, 160. By way of example and not limitation, the attachment means may comprise screws 145, 175 adapted to extend through an aperture with a grommet 140, 170 in the instrument tray 105 and thread into a threaded holes 155, 165 in the vertical support. Tightening the screws 145, 175 secures the instrument tray 105 to the vertical supports 150, 160. Removal of the screws 145, 175 allows disassembly of the sterility maintenance cover and removal of the instrument tray 105. Although not shown in FIG. 2, the bottom end of the vertical supports 150, 160 may be removably attached to the base panel in a similar manner. Other forms of permanent or releasable attachment may be utilized in addition to or in lieu of screws.

Referring now to FIG. 3, an exemplary modular sterility maintenance cover with support flanges in accordance with principles of the invention is conceptually shown. This embodiment is similar to the embodiment depicted in FIG. 2, except it includes a plurality of support flanges 180, 185 for enhanced structural support. The sterility maintenance cover generally includes a base panel 110 configured to provide a stable support surface. A plurality of vertical supports 150, 160 extend from the base panel 110 to a height sufficient to clear a patient's head or other anatomical structure. The support flanges 180, 185 extend outwardly from the top end of vertical supports 150, 160, providing support structures that abut the underside of the instrument tray 105. The instrument tray 105 is removably attached to the vertical supports 150, 160, at the end opposite the base panel 110, atop the support flanges 180, 185. The periphery of the topside of the instrument tray 105 includes raised edges 115-135 to prevent objects from sliding off.

Attachment means is provided to removably attach the instrument tray 105 to the vertical supports 150, 160. By way of example and not limitation, the attachment means may comprise screws 145, 175 adapted to extend through an aperture with a grommet 140, 170 in the instrument tray 105 and thread into a threaded holes 155, 165 in the vertical support. Tightening the screws 145, 175 will secure the instrument tray 105 to the vertical supports 150, 160. Removal of the screws 145, 175 allows disassembly of the sterility maintenance cover and removal of the instrument tray 105. Other forms of permanent or releasable attachment may be utilized in addition to or in lieu of screws.

Likewise, attachment means is provided to removably attach the base panel 110 to the vertical supports 150, 160. By way of example and not limitation, the attachment means may comprise screws 190, 195 adapted to extend through an aperture in the base panel 110 and thread into a threaded holes 200, 205 in the vertical supports. Tightening the screws 190, 195 will secure the base panel 110 to the vertical supports 150, 160. Removal of the screws 190, 195 allows disassembly of the sterility maintenance cover and removal of the base panel 110. Other forms of permanent or releasable attachment may be utilized in addition to or in lieu of screws.

Referring now to FIG. 4, an exemplary modular sterility maintenance cover with support flanges in accordance with principles of the invention is conceptually shown. This embodiment is similar to the embodiment depicted in FIG. 2, except it includes a plurality of clamps 470, 485 adapted for releasably engaging an edge of the instrument tray 105. The sterility maintenance cover generally includes a base panel 410 configured to provide a stable support surface. A plurality of vertical supports 450, 460 extend from the base panel 410 to a height sufficient to clear a patient's head or other anatomical structure. The instrument tray 405 is removably attached to the vertical supports 450, 460, at the end opposite the base panel 410, using a plurality of clamps 470, 485 adapted for releasably engaging an edge 435 of the instrument tray 105. The exemplary clamps 470, 485 include protrusions 475, 490 to resist removal of an engaged instrument tray 105. The exemplary clamps 470, 485 also include flat surfaces 465, 480 upon which the bottom of the engaged portions of the tray 105 will rest. The periphery of the topside of the instrument tray 105 includes raised edges 415-430 to prevent objects from sliding off and a raised edge 435 to facilitate engagement by the clamps 470, 485. Other types of clamps, such as clamps with spring and/or threaded actuation may be utilized in addition to or in lieu of clamps 470, 485. Such other types of clamps come within the scope of the invention.

Attachment means may be provided to removably attach the base panel 410 to the vertical supports 450, 460. By way of example and not limitation, the attachment means may comprise screws (not shown in FIG. 4) adapted to extend through an aperture in the base panel and thread into a threaded holes in the vertical supports. Tightening the screws secures the base panel 410 to the vertical supports. Removal of the screws, allows disassembly of the sterility maintenance cover and removal of the base panel. Other forms of permanent or releasable attachment may be utilized in addition to or in lieu of screws.

Referring now to FIG. 7, an exemplary sterility maintenance cover with an adjustable height tray for engaging a removable tray in accordance with principles of the invention is conceptually shown. This embodiment is similar to the embodiment depicted in FIG. 3, except it includes height adjustment means. The sterility maintenance cover generally includes a base panel 110 configured to provide a stable support surface. A plurality of vertical supports 150, 160 extend from the base panel 110 to a height sufficient to clear a patient's head or other anatomical structure. The support flanges 180, 185 extend outwardly from the top end of vertical supports 150, 160, providing support structures that abut the underside of the instrument tray 105. The instrument tray 105 is removably attached to the vertical supports 150, 160, at the end opposite the base panel 110, atop the support flanges 180, 185. The periphery of the topside of the instrument tray 105 includes raised edges 115-135 to prevent objects from sliding off.

The height adjustment means may be comprised of any manually operated linear actuator or other manually controlled adjustable length support devices. A non-limiting example includes a threaded shaft 715, 730 which is adapted for threading into a correspondingly threaded tube 710, 725. The threaded tube 710, 725 may be adapted to bi-directionally rotate. Rotation of the threaded tube 710, 725 in one direction causes the threaded shaft 715, 730 to emerge from the threaded tube, thereby extending the overall length of the vertical supports comprised of top section 735, 740, threaded shaft 715, 730, threaded tube 710, 725 and bottom section 705, 720. Rotation of the threaded tube 710, 725 in the other direction causes the threaded shaft 715, 730 to return into the threaded tube, thereby reducing the overall length of the vertical supports comprised of top section 735, 740, threaded shaft 715, 730, threaded tube 710, 725 and bottom section 705, 720. Other threaded and telescopic extension and retraction apparatus may be utilized without departing from the scope of the invention.

Optionally, a spirit level 750 or bubble level may be provided on the tray 105 to indicate whether tray is level. Such a level features a glass tube which is incompletely filled with a liquid, usually colored ethanol, leaving a bubble in the tube. The level 750 will indicate if one vertical support has been adjusted appreciably higher than the other vertical support.

Attachment means is provided to removably attach the instrument tray 105 to the vertical supports 150, 160. By way of example and not limitation, the attachment means may comprise screws 145, 175 adapted to extend through an aperture with a grommet 140, 170 in the instrument tray 105 and thread into a threaded holes 155, 165 in the vertical support. Tightening the screws 145, 175 secures the instrument tray 105 to the vertical supports 150, 160. Removal of the screws 145, 175 allows disassembly of the sterility maintenance cover and removal of the instrument tray 105. Other forms of permanent or releasable attachment may be utilized in addition to or in lieu of screws.

Likewise, attachment means is provided to removably attach the base panel 110 to the vertical supports 150, 160. By way of example and not limitation, the attachment means may comprise screws 190, 195 adapted to extend through an aperture in the base panel 110 and thread into a threaded holes 200, 205 in the vertical supports. Tightening the screws 190, 195 secures the base panel 110 to the vertical supports 150, 160. Removal of the screws 190, 195 allows disassembly of the sterility maintenance cover and removal of the base panel 110. Other forms of permanent or releasable attachment may be utilized in addition to or in lieu of screws.

Referring now to FIG. 8A, an exemplary modular sterility maintenance cover in accordance with principles of the invention is shown. This embodiment is similar to the embodiment depicted in FIG. 2, except the base panel 110 and tray 105 are hingedly attached to the vertical supports 810, 815. The exemplary sterility maintenance cover includes a plurality of vertical supports 810, 815 for structural integrity. The sterility maintenance cover generally includes a base panel 110 configured to provide a stable support surface. The vertical supports 810, 815 extend from the hingedly attached base panel 110 to a height sufficient to clear a patient's head or other anatomical structure. The instrument tray 105 is hingedly attached to the vertical supports 810, 815 at the end opposite the base panel 110. The periphery of the topside of the instrument tray 105 includes raised edges 115-135 to prevent objects from sliding off.

Hinges 800, 805, 820, 825 are provided to enable the base panel 110 and tray 105 to swing between a substantially flat storage orientation with the base panel 110 and tray 105 folded against the backs of the vertical supports 810, 815 (as shown in FIG. 8B) to an operational orientation with the base panel 110 and tray 105 extending perpendicular to the longitudinal axis of the vertical supports 810, 815 (as shown in FIG. 8A). The storage orientation facilitates storage, packaging and shipping of the sterility maintenance cover. Pivot hinges, butt/mortise hinges, continuous hinges(also known as a “piano hinges”), concealed hinges or any other compatible hinge assemblies, with or without dampening systems may be utilized.

Attachment means is provided to removably attach the instrument tray 105 to the vertical supports 150, 160. By way of example and not limitation, the attachment means may comprise screws 145, 175 adapted to extend through an aperture with a grommet 140, 170 in the instrument tray 105 and thread into a threaded holes 155, 165 in the vertical support. Tightening the screws 145, 175 secures the instrument tray 105 to the vertical supports 150, 160. Removal of the screws 145, 175 allows disassembly of the sterility maintenance cover and removal of the instrument tray 105. Although not shown in FIG. 2, the bottom end of the vertical supports 150, 160 may be removably attached to the base panel in a similar manner. Other forms of permanent or releasable attachment may be utilized in addition to or in lieu of screws.

The base panels, vertical supports and instrument trays in the embodiments shown in FIGS. 1-4 and 6-8 may be constructed of plastic materials, and preferably transparent radiolucent plastic materials. The base panels, vertical supports and instrument trays are preferably sufficiently strong and moisture resistant to maintain integrity while subjected to the stresses and environmental conditions encountered during use. In an exemplary implementation, the base 110 is comprised of a rigid plastic or polymeric material, such as polyvinyl chloride (PVC), polyethylene, polypropylene, polystyrene, acrylics, cellulosics, acrylonitrile-butadiene-styrene terpolymers, urethanes, thermo-plastic resins, thermo-plastic elastomers (TPE), acetal resins, polyamides, polycarbonates and polyesters. While many other materials may be used alone or in combination with the aforementioned materials and/or other materials, without departing from the scope of the present invention, preferably the material is relatively inexpensive, easy to use in manufacturing operations and results in an aesthetically acceptable, durable product. The material may further include additives to provide desired properties such as desired colors, structural characteristics, glow-in-the dark properties and thermal reactivity (e.g., color changes according to heat). Screws and any other components and accessories may also be comprised of radiolucent materials.

By way of example and not limitation, components of the sterility maintenance cover may optionally be formulated to change color when it reaches a predetermined or higher temperature. This can be accomplished by mixing a thermochromic additive to the base material in an amount that is sufficient to achieve a desired color changing range. As an example, a mixture of approximately 5% to 30% (pbw) of Matsui International Co., Inc.'s Chromicolor® concentrate may be introduced to the base material, to provide a plastic structure that visibly changes color at a determined elevated temperature, such as approximately 90 degrees Fahrenheit or higher.

Alternatively, a photochromic additive may be added to the base material in an amount that is effective to achieve a desired color change when the base 100 is exposed to certain lighting conditions. As an example, a mixture of approximately 5% to 35% (pbw) of Matsui International Co., Inc.'s Photopia® additive may be introduced to the base material, to provide a plastic structure that visibly changes color in the presence of sunlight or ultraviolet light.

As another alternative, phosphorescent polymer additives, such as aluminate based phosphors, may be added to adsorb light energy and continue to release that energy as visible light after the energy source is removed. Advantageously, such an embodiment provides a structure that is easy to locate in darkened conditions.

The sterility maintenance cover may be produced using any suitable manufacturing techniques known in the art for the chosen material, such as (for example) injection, compression, structural foam, blow, or transfer molding; polyurethane foam processing techniques; vacuum forming; casting and milling. Preferably, the manufacturing technique is suitable for mass production at relatively low cost per unit, and results in an aesthetically acceptable product with a consistent acceptable quality.

Another exemplary sterility maintenance cover in accordance with the principles of the invention may be constructed by folding a one-piece blank 500, which is conceptually illustrated in FIG. 5. The exemplary blank 500 is die-cut and scored, according to known techniques, from a flat sheet of laminated material, examples of which are described in greater detail below. Other cutting techniques can be used, such as knife-cutting, razor-cutting, and the like. Advantageously, the blank 500 may be conveniently shipped and stored as a flat structure.

The blank 500 has four panels 505, 510, 515 and 520 each of which is defined by a generally square area of the blank 500. As shown in FIG. 6, panel 505 corresponds to the instrument tray 505; panel 510 corresponds to a vertical support 510; panel 515 corresponds to the base panel 515; and panel 520 corresponds to a vertical support 520.

Each vertical support 510, 520 is formed by folding the vertical support panels 510, 520 along the horizontal fold lines 560, 565 and 605, 620 and along vertical fold lines 555, 575 and 600, and then slipping tabs into corresponding slots. To form one vertical support between the instrument tray 505 and base panel 515, tabs 550 and 545 are slipped into slots 540 and 535 respectively, and tabs 570 and 572 are slipped into slots 585 and 580 respectively. To form another vertical support between the instrument tray 505 and base panel 515, tabs 610 and 615 are slipped into slots 595 and 590 respectively, and tabs 625 and 630 are slipped into slots 525 and 530 respectively.

The sterility maintenance cover thus formed from the blank is conceptually shown in FIG. 6. A pair of C-channel beams comprise the vertical supports. The vertical supports extend from the base panel 515 to the instrument tray 505. It will be understood by those of ordinary skill in the art that the vertical supports are of similar construction, and differ only in position, as shown in the exemplary embodiment depicted in the drawing figures.

While the exemplary sterility maintenance cover depicted in FIG. 6 is preferably constructed from a flat, die-cut blank, it will understood by those of ordinary skill in the art that a generally flat, contiguous blank may be pared down through knife cuts, razor blade cuts, and the like, to produce the various configurations of blank 500 described herein. Additionally, the blank 500, shown best by FIG. 5, may be fabricated from a laminate material comprising plastic films bonded to a plastic or paperboard, or corrugated plastic or paperboard substrate. In the preferred embodiment, the laminated plastic or paperboard substrate is an E-fluted corrugated plastic or paperboard, but other materials may be substituted, for example, A-, B-, C-, D-, E, or F-fluted, or micro-corrugated paperboard, or other foldable materials. Lamination of the substrate can be completed before or after fluting, but lamination should be completed before scoring and cutting the blank 500. An extruded plastic material, such as Coroplast®, which consists of top and bottom faces, connected by multiple parallel ribs, is another suitable material.

Optionally, an instrument tray 105, 505 according to principles of the invention may include an absorbent pad overlay, for purposes of soaking up any liquid waste deposited on the tray during a procedure. The absorbent pad may be bonded to the tray surface; stitched to the tray surface, or releasably attached using tape, adhesive backing, hook and loop fasteners or other releasable attachment means.

While an exemplary embodiment of the invention has been described, it should be apparent that modifications and variations thereto are possible, all of which fall within the true spirit and scope of the invention. With respect to the above description then, it is to be realized that the optimum relationships for the components of the invention and steps of the process, including variations in form, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A sterility maintenance cover comprising a base panel, a first vertical support having a bottom end and a top end and being attached to the base panel at the bottom end extending upwardly from the from the base panel to a height sufficient to clear a patient's anatomical structure, an instrument tray removably attached to the first vertical support at the top end, said instrument tray having a topside with a periphery having raised edges.

2. A sterility maintenance cover according to claim 1, further comprising attachment means adapted to removably and pivotally attach the instrument tray to the first vertical support.

3. A sterility maintenance cover according to claim 2, said attachment means comprising a screw adapted to extend through an aperture with a grommet in the instrument tray and thread into a threaded hole in the top end of the first vertical support.

4. A sterility maintenance cover according to claim 1, further comprising a support flange extending outwardly from the top end of the first vertical support and being configured to abut and provide support to the instrument tray.

5. A sterility maintenance cover according to claim 1, further comprising a second vertical support having a bottom end and a top end and being attached to the base panel at the bottom end extending upwardly from the from the base panel to a height sufficient to clear a patient's anatomical structure, said second vertical support structure being a determined distance from the first vertical support structure.

6. A sterility maintenance cover according to claim 5, further comprising attachment means adapted to removably and pivotally attach the instrument tray to the first vertical support and second vertical support.

7. A sterility maintenance cover according to claim 5, further comprising a plurality of support flanges, at least one of said plurality of support flanges extending outwardly from the top end of each of the first and second vertical supports and being configured to abut and provide support to the instrument tray.

8. A sterility maintenance cover according to claim 5, said instrument tray including at least one edge, and said sterility maintenance cover further comprising a clamp attached to the top end of each support, said clamp being adapted to securely releasably engage an edge of the instrument tray.

9. A sterility maintenance cover according to claim 8, said clamp including a protrusion configured to resist removal of the instrument tray after engagement.

10. A sterility maintenance cover according to claim 9, further including a height adjustment means adapted to enable manual adjustment of the length of each of said first and second vertical supports.

11. A sterility maintenance cover according to claim 10, wherein the height adjustment means comprises a manually adjustable first threaded shaft operably coupled to said first vertical support, and a manually adjustable second threaded shaft operably coupled to said second vertical support.

12. A sterility maintenance cover according to claim 10, further including a level indicator operably coupled to the instrument tray.

13. A sterility maintenance cover according to claim 12, wherein said level indicator comprises a spirit level.

14. A sterility maintenance cover according to claim 1, wherein the base panel, first vertical support and instrument tray are comprised of a transparent radiolucent plastic.

15. A sterility maintenance cover according to claim 5, wherein the base panel, first vertical support, second vertical support and instrument tray are comprised of a transparent radiolucent plastic.

16. A sterility maintenance cover according to claim 5, wherein the base panel, first vertical support, second vertical support and instrument tray include an additive from the group consisting of thermochromic, photochromic and phosphorescent additives.

17. A sterility maintenance cover comprising a base panel, an instrument tray, a first vertical support having a bottom end and a top end, a second vertical support having a bottom end and a top end, a plurality of hinged attachments, said base panel being hingedly attached to the bottom end of said first and second vertical supports and said instrument tray being hingedly attached to the top end of said first and second vertical supports, said tray having a topside with a periphery having raised edges.

18. A sterility maintenance cover according to claim 14, wherein said base panel is adapted to hingedly pivot between an operational configuration and a storage configuration, said operational configuration being weight-bearing and perpendicular to the longitudinal axes of the first and second vertical supports, and said instrument tray is adapted to hingedly pivot between an operational configuration and a storage configuration, said operational configuration being weight-bearing and perpendicular to the longitudinal axes of the first and second vertical supports.

19. A sterility maintenance cover comprised of a base panel formed from a first panel of an integral blank, a first vertical support formed from a second panel of the integral blank, said first vertical support having a bottom end and a top end, an instrument tray formed from a third panel of the integral blank, and a second vertical support formed from a fourth panel of the integral blank, said second vertical support having a bottom end and a top end, said first panel of the integral blank being attached to said said second panel of the integral blank along a first edge of the second panel, said second panel of the integral blank being attached to said third panel of the integral blank along a second edge of the second panel, said second edge being opposite said first edge, and said third panel of the integral blank being attached to said fourth panel of the integral blank along a first edge of the fourth panel, said first edge of the fourth panel being opposite the second edge of the second panel.

20. A sterility maintenance cover according to claim 19, said integral blank comprising a material from the group consisting of corrugated plastic, corrugated paperboard, fluted corrugated plastic, fluted corrugated paperboard, and micro-corrugated paperboard.
